(19) United States
(12) Patent Application Publication
(10) Pub. No.: US 2007/0065392 A1
(43) Pub. Date: Mar. 22, 2007

(54) Cosmetic composition with an amphiphilic lipid phase

(75) Inventor: Jean-Thierry Simonnet, Cachan (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/484,659

(22) Filed: Jul. 12, 2006

Related U.S. Application Data
(60) Provisional application No. 60/704,418, filed on Aug. 2, 2005.

(30) Foreign Application Priority Data
Jul. 12, 2005 (FR) 05 52163

(51) Int. Cl. A61K 8/42

(52) U.S. Cl. 424/70.31

(57) ABSTRACT

A cosmetic makeup and/or care composition, including at least one amphiphilic lipid phase organized as a lamellar or cubic liquid crystal phase, wherein the lipid phase includes at least one urea-based compound of formula (I) 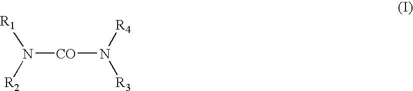
or a salt, solvate, or isomer thereof, wherein R₁, R₂, R₃, and R₄ each independently represent a hydrogen atom, a C₁-C₄ alkyl group, or a C₂-C₆ hydroxyalkyl group that includes from 1 to 5 hydroxyl groups, wherein at least one of the radicals R₁ to R₄ represents a hydroxyalkyl group, the urea-based compound being present in the amphiphilic phase.

### CROSS-REFERENCE TO RELATED APPLICATIONS

This non provisional application claims the benefit of French Application No. 05 52163 filed on Jul. 12, 2005 and U.S. Provisional Application No. 60/704,418 filed on Aug. 2, 2005, the disclosures of which are incorporated herein by reference in their entireties.

### BACKGROUND

It is known that certain amphiphilic lipids have the property of forming mesomorphous phases, an intermediate organizational state between the crystal state and the liquid state, in lamellar, cubic, or hexagonal phases.

With respect to lamellar phases, they have a stacking-type structure of bimolecular layers. When a lamellar phase is dispersed in an excess of water, this lamellar phase may then form vesicles. The vesicles thus obtained are in the form of spherules whose outer membrane has a lamellar structure consisting of one or more lipid layers, separated from each other by layers of aqueous phase. The lipid composition that may be used to prepare these spherules may be of an ionic nature and, in this case, liposomes are obtained, or may be of a non-ionic nature, and niosomes are then obtained.

Oil-in-water emulsions whose oily globules are coated with amphiphilic lipids organized in a lamellar liquid crystal phase and located at the oil/aqueous phase interface are also known.

For its part, a cubic liquid crystal phase is organized in a bipolar manner as separate hydrophilic and lipophilic domains, in close and organized contact in a three-dimensional network. This cubic liquid crystal phase is generally in the form of a gel that may also be dispersed to form particles. The dispersed particles of cubic liquid crystal phase have a lamellar outer structure and a bi-continuous cubic inner structure. This inner structure has separate hydrophilic and lipophilic domains. These particle dispersions are especially advantageous for stabilizing an oily phase in water-in-oil systems.

In general, supports based on a lamellar liquid crystal phase, for instance liposomes, niosomes or oleosomes, or based on cubic gel, are particularly appreciated for their moisturizing properties and their reinforcement of the barrier function of keratin materials and especially of the skin. Moreover, since the liquid crystal phases have hydrophilic and hydrophobic domains, they are particularly advantageous for dissolving or dispersing a wide variety of hydrophilic, lipophilic and amphiphilic compounds.

In all cases, the amphiphilic lipids constituting a liquid crystal phase must be swollen (or hydrated) in order to form a lamellar or cubic liquid crystal phase. This is usually performed with water or glycols (glycerol, propylene glycol, butylene glycol, dipropylene glycol, etc.). However, the moisturizing potential of a lamellar phase hydrated with water is insufficient. The addition of water to the aqueous hydrating phase or even the hydration of lipids with a pure glycolic phase significantly improves these moisturizing properties. However, this is accompanied by an undeniable tacky effect that a person skilled in the art seeks to minimize.

There is thus still a need to hydrate amphiphilic lipids, which are capable of forming a lamellar or cubic liquid crystal phase, with a derivative that is at least as effective as glycols and much less tacky, to exploit the moisturizing properties of such structures.

### SUMMARY

It has now been found that the addition of a particular urea derivative allows suitable hydration of amphiphilic lipids organized as a lamellar or cubic liquid crystal phase by overcoming the drawbacks mentioned previously, especially in terms of the tacky effect associated with the presence of glycols such as glycerol.

The present invention relates to a composition comprising at least one amphiphilic lipid phase organized as a lamellar or cubic liquid crystal phase and also comprising at least one urea derivative.

A first subject of the present invention is thus a cosmetic makeup and/or care composition, especially for keratin material(s), comprising at least one amphiphilic lipid phase organized as a lamellar or cubic liquid crystal phase, characterized in that it also comprises at least one urea-based compound of formula (I) 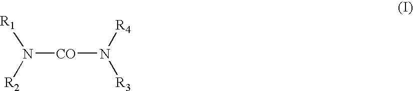
in which R₁, R₂, R₃, and R₄ each independently represent a hydrogen atom, a C₁-C₄ alkyl group or a C₂-C₆ hydroxyalkyl group that may contain from 1 to 5 hydroxyl groups, in which at least one of the radicals R₁ to R₄ represents a hydroxyalkyl group, or a salt, solvate, or isomer thereof, the one urea based compound being present in the amphiphilic liquid phase.

In a first variant, the composition according to the invention comprises an amphiphilic lipid phase organized as a lamellar liquid crystal phase referred to as “phase A” swollen with at least one urea derivative of formula (I) as defined previously.

According to a first embodiment of this variant, the composition comprises an aqueous dispersion of lipid vesicles comprising phase A and in particular an aqueous dispersion of liposomes or niosomes.

According to a second embodiment, the composition is in the form of an oil-in-water emulsion comprising vesicles with an oily core having a coating consisting of phase A.

According to a third embodiment, the composition is essentially anhydrous and comprises at least one fatty phase that is especially liquid and separate from the lamellar liquid crystal phase in which is dispersed phase A.

For the purposes of the present invention, an “essentially anhydrous” composition means a composition according to the invention containing less than 7% by weight of water, in particular less than 5% by weight, more particularly less than 1% by weight, or even less than 0.5% by weight of water.

In a second variant, the composition comprises an aqueous dispersion of particles of a cubic gel comprising at least one urea derivative of formula (I) as defined previously.

According to one particular embodiment of this variant, this composition is in the form of an oil-in-water dispersion stabilized by the particle dispersion.

According to another of its aspects, a subject of the present invention is also a cosmetic process for treating keratin materials, in particular the skin, especially a non-therapeutic makeup and/or care process comprising at least the application of a cosmetic composition as defined previously to the keratin materials.

### DETAILED DESCRIPTION OF EMBODIMENTS

Urea Derivatives

The urea compound present in the composition according to the invention is a compound of formula (I) below: 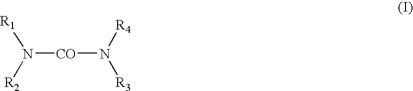
in which R₁, R₂, R₃, and R₄ represent, independently of each other, a hydrogen atom, a C₁-C₄ alkyl group or a C₂-C₆ hydroxyalkyl group possibly containing from 1 to 5 hydroxyl groups, in which at least one of the radicals R₁ to R₄ represents a hydroxyalkyl group, and also the salts, solvates, and isomers thereof.

For the compounds of formula (I), preferably, R₁ denotes a C₂-C₆ hydroxyalkyl group and R₂, R₃, and R₄ denote, independently of each other, a hydrogen atom or a C₁-C₄ alkyl group. Preferentially, R₁ denotes a C₂-C₆ hydroxyalkyl group comprising from 1 to 5 hydroxyl groups and especially one hydroxyl group, and R₂, R₃, and R₄ denote a hydrogen atom. More preferentially, R₁ denotes a C₂-C₄ hydroxyalkyl group comprising one hydroxyl group and R₂, R₃, and R₄ denote a hydrogen atom.

Alkyl groups that may especially be mentioned include methyl, ethyl, n-propyl, isopropyl, n-butyl, isobutyl, and tert-butyl groups.

Among the hydroxyalkyl groups that are preferred are those containing only one hydroxyl group and in particular hydroxyethyl, hydroxypropyl, hydroxybutyl, hydroxypentyl, and hydroxyhexyl groups.

Salts of such compounds that may be mentioned include the salts of mineral acids, such as sulfuric acid, hydrochloric acid, hydrobromic acid, hydriodic acid, phosphoric acid, and boric acid. Mention may also be made of the salts of organic acids, which may comprise one or more carboxylic acid, sulfonic acid, or phosphonic acid groups. They may be linear, branched, or cyclic aliphatic acids or alternatively aromatic acids. These acids may also comprise one or more hetero atoms chosen from O and N, for example in the form of hydroxyl groups. Mention may especially be made of propionic acid, acetic acid, terephthalic acid, citric acid, and tartaric acid.

The term “solvate” means a stoichiometric mixture of the said compound of formula (I) with one or more water molecules or organic solvent molecules, such a mixture being derived from the synthesis of the compound of formula (I).

Preferred compounds of formula (I) that may be mentioned include N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof. Preferably, the compound of formula (I) is N-(2-hydroxyethyl)urea.

The compounds of formula (I) are known compounds and are described especially in patent application DE-A-2 703 185. Among these, N-(2-hydroxyethyl)urea is also commercially available, in the form of a mixture at 50% by weight in water, from the company National Starch under the trade name Hydrovance®.

The compound of formula (I) is present in an effective amount, i.e. in an amount that is sufficient to give the composition into which it is incorporated the desired moisturizing effect.

More specifically, the compound of formula (I) may be present in the compositions according to the invention in content ranging from 1% to 50% by weight, preferably ranging from 2% to 25% by weight and preferentially ranging from 2% to 20% by weight, relative to the total weight of the composition.

Advantageously, the compound of formula (I) is conveyed in an aqueous phase prior to being mixed with the amphiphilic lipid phase.

Amphiphilic Lipid Phase

For purposes of the present invention, the term “amphiphilic lipid phase” means a phase composed of amphiphilic lipid or mixture of amphiphilic lipids.

The term “amphiphilic lipid” means herein any molecule having a bipolar structure, i.e. comprising at least one hydrophobic portion and at least one hydrophilic portion having the property of reducing the interface tension between water and an oily phase. The synonyms of amphiphilic lipid are, for example: surfactant, surface agent, and emulsifier.

With respect to amphiphilic lipids that may be used to form the lipid phase organized in a lamellar or cubic liquid crystal phase, they may be any amphiphilic lipid known for its capacity to form a lamellar phase in the presence of water. These lipids are, in a known manner, ionic or non-ionic amphiphilic lipids of natural or synthetic origin, comprising, per molecule, one or more linear or branched, saturated or unsaturated long hydrocarbon-based chain(s), preferably containing 8 to 30 carbon atoms, these chains being, for example, an oleic, lanolic, tetradecyl, hexadecyl, isostearyl, lauryl or alkylphenyl chain, and one or more hydrophilic group(s) taken from among hydroxyl, oxide ether, carboxyl, phosphate, and amine groups.

The amphiphilic lipid phase generally comprises at least one non-ionic amphiphilic lipid and, where appropriate, an anionic amphiphilic lipid.

The non-ionic amphiphilic lipids of the invention that are most particularly suitable are chosen from:
1) silicone surfactants,
2) amphiphilic lipids that are liquid at a temperature of less than or equal to 45° C., chosen from esters of at least one polyol of at least one fatty acid containing at least one saturated or unsaturated, linear or branched, and especially unsaturated or branched, C₈-C₂₂ alkyl chain, the polyol being chosen from the group formed by polyethylene glycol comprising from 1 to 60 ethylene oxide units, sorbitan, glycerol possibly comprising from 2 to 30 ethylene oxide units, and polyglycerols comprising from 2 to 15 glycerol units,
3) fatty acid esters of sugars and fatty alkyl ethers of sugars,
4) surfactants that are solid at a temperature equal to 45° C., chosen from fatty esters of glycerol, fatty esters of sorbitan and oxyethylenated fatty esters of sorbitan, ethoxylated fatty ethers and ethoxylated fatty esters,
5) block copolymers of ethylene oxide (A) and of propylene oxide (B), and mixtures thereof,
6) phospholipids (natural or synthetic), for instance phosphatidylcholine.

As illustrations of silicone surfactants that may be used according to the invention, mention may be made especially of silicone compounds comprising at least one oxyethylene —OCH₂CH₂— and/or oxypropylene —OCH₂CH₂CH₂— chain. Silicone surfactants that may be used according to the present invention, mention may be made of those described in documents U.S. Pat. No. 5,364,633 and U.S. Pat. No. 5,411,744.

In particular, the silicone surfactant used according to the present invention may be a compound of formula (II): 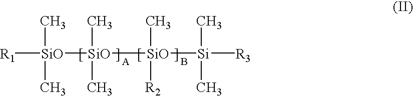
in which R₁, R₂, and R₃, independently of each other, represent a C₁-C₆ alkyl radical or a radical —(CH₂)ₙ—(OCH₂CH₂)_{y}—(OCH₂CH₂CH₂)_{z}—OR₄, at least one radical R₁, R₂, or R₃ not being an alkyl radical; R₄ being a hydrogen, an alkyl radical or an acyl radical; A is an integer ranging from 0 to 200; B is an integer ranging from 0 to 50; with the condition that A and B are not simultaneously equal to zero; x is an integer ranging from 1 to 6; y is an integer ranging from 1 to 30; and z is an integer ranging from 0 to 5.

According to one preferred embodiment of the invention, in the compound of formula (X), the alkyl radical is a methyl radical, x is an integer ranging from 2 to 6, and y is an integer ranging from 4 to 30.

Examples of silicone surfactants of formula (II) that may be mentioned include the compounds of formula (II): 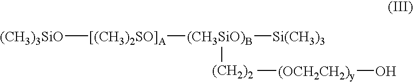
in which A is an integer ranging from 20 to 105, B is an integer ranging from 2 to 10, and y is an integer ranging from 10 to 20.

Examples of silicone surfactants of formula (II) that may also be mentioned include the compounds of formula (IV):
H—(OCH₂CH₂)_{y}—(CH₂)₃—[(CH₃)₂SiO]_{A}—(CH₂)₃—(OCH₂CH₂)_{y}—OH (IV)
in which A′ and y are integers ranging from 10 to 20.

Silicone surfactants such as those sold by the company Dow Corning under the names DC 5329, DC 7439-146, DC 2-5695 and Q4-3667 may be used in particular. The compounds DC 5329, DC 7439-146 and DC 2-5695 are compounds of formula (III) in which, respectively, A is 22, B is 2, and y is 12; A is 103, B is 10, and y is 12; A is 27, B is 3, and y is 12.

The amphiphilic lipids that are liquid at a temperature of less than or equal to 45° C. may be chosen especially from polyethylene glycol isostearate of molar weight 400 (CTFA name: PEG-8 isostearate), sold under the name Prisorine 3644 by the company Uniqema; diglyceryl isostearate, especially the product sold by the company Solvay; polyglyceryl laurate comprising 2 glycerol units (polyglyceryl-2 laurate), especially the product sold under the name diglycerin monolaurate by the company Solvay; sorbitan oleate, especially the product sold under the name SPAN 80 by the company ICI; sorbitan isostearate, especially the product sold under the name Nikkol SI 10R by the company Nikkol; α-butylglucoside cocoate or α-butylglucoside caprate sold especially by the company Ulice.

The fatty acid esters of sugars that may be used as non-ionic amphiphilic lipids according to the invention are preferably solid at a temperature of less than or equal to 45° C. and may be chosen especially from the group comprising esters or mixtures of esters of C₈-C₂₂ fatty acids and of sucrose, maltose, glucose or fructose, and esters or mixtures of esters of C₁₄-C₂₂ fatty acids and of methylglucose. The C₈-C₂₂ or C₁₄-C₂₂ fatty acids forming the fatty unit of the esters that may be used comprise a saturated or unsaturated linear alkyl chain, of 8 to 22 or of 14 to 22 carbon atoms, respectively. The fatty unit of the esters may be chosen especially from stearates, behenates, arachidonates, palmitates, myristates, laurates, caprates, and mixtures thereof. Stearates are especially used.

Examples of esters or mixtures of esters of fatty acid and of sucrose, maltose, glucose, or fructose that may be mentioned include sucrose monostearate, sucrose distearate, sucrose tristearate, and mixtures thereof, such as the products sold by the company Croda under the name Crodesta F50, F70, F110, and F160, respectively having an HLB (Hydrophilic-Lipophilic Balance) of 5, 7, 11, and 16; and an example of esters or mixtures of esters of fatty acid and of methylglucose that may be mentioned is methylglucose polyglyceryl-3 distearate, sold by the company Goldschmidt under the name Tegocare 450. Mention may also be made of monoesters of glucose or of maltose such as methyl O-hexadecanoyl-6-D-glucoside and O-hexadecanoyl-6-D-maltoside.

The sugar fatty alcohol ethers that may be used as non-ionic amphiphilic lipids are solid at a temperature of less than or equal to 45° C. and may be chosen especially from the group comprising ethers or mixtures of ethers of C₈-C₂₂ fatty alcohol and of glucose, maltose, sucrose, or fructose, and ethers or mixtures of ethers of C₁₄-C₂₂ fatty alcohol, and of methylglucose. They are especially alkylpolyglucosides.

The C₈-C₂₂ or C₁₄-C₂₂ fatty alcohols forming the fatty unit of the ethers that may be used according to the invention comprise a saturated or unsaturated linear alkyl chain containing, respectively, from 8 to 22 or from 14 to 22 carbon atoms. The fatty unit of the ethers may be chosen especially from decyl, cetyl, behenyl, arachidyl, stearyl, palmityl, myristyl, lauryl, carry, and hexadecanoyl units, and mixtures thereof such as cetearyl.

Examples of sugar fatty alcohol ethers that may be mentioned include alkylpolyglucosides such as decyl glucoside and lauryl glucoside sold, for example, by the company Henkel under the respective names Plantaren 2000 and Plantaren 1200, cetostearyl glucoside optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidyl glucoside, for example in the form of the mixture of arachidyl and behenyl alcohols and of arachidyl glucoside, sold under the name Montanov 202 by the company SEPPIC.

Sucrose monostearate, sucrose distearate, sucrose tristearate, and mixtures thereof, methylglucose polyglyceryl-3 distearate, and alkylpolyglucosides are more particularly used as non-ionic amphiphilic lipid of this type.

The fatty esters of glycerol that may be used as non-ionic amphiphilic lipids according to the invention, which are solid at a temperature equal to 45° C., may be chosen especially from the group comprising the esters formed from at least one acid comprising a saturated linear alkyl chain containing from 16 to 22 carbon atoms and from 1 to 10 glycerol units. These esters may be chosen especially from stearates, behenates, arachidates, palmitates, and mixtures thereof. Stearates and palmitates are preferably used.

Examples of surfactants of this type that may be mentioned include decaglyceryl monostearate, distearate, tristearate, and pentastearate (10 glycerol units) (CTFA names: polyglyceryl-10 stearate, polyglyceryl-10 distearate, polyglyceryl-10 tristearate and polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate), such as the product sold by the company Nikko under the name Nikkol DGMS.

The fatty esters of sorbitan that may be used as non-ionic amphiphilic lipids according to the invention, which are solid at a temperature equal to 45° C., are chosen especially from the group comprising esters of C₁₆-C₂₂ fatty acid and of sorbitan and oxyethylenated esters of C₁₆-C₂₂ fatty acid and of sorbitan. They are formed from at least one fatty acid comprising at least one saturated linear alkyl chain containing, respectively, from 16 to 22 carbon atoms and from sorbitol or ethoxylated sorbitol. The oxyethylenated esters generally comprise from 1 to 100 ethylene oxide units and preferably from 2 to 40 ethylene oxide (EO) units. These esters may be chosen especially from stearates, behenates, arachidates, palmitates, and mixtures thereof. Stearates and palmitates are preferably used.

As examples of fatty esters of sorbitan and of oxyethylenated fatty esters or sorbitan that may be used according to the invention, mention may be made of sorbitan monostearate CTFA name: sorbitan stearate) sold by the company ICI under the names Span 60, sorbitan monopalmitate (CTFA name: sorbitan palmitate) sold by the company ICI under the name Span 40, and sorbitan 20 EO tristearate (CTFA name: polysorbate 65) sold by the company ICI under the name Tween 65.

The ethoxylated fatty ethers that are solid at a temperature equal to 45° C., which may be used as non-ionic amphiphilic lipids that may be used according to the invention, are preferably ethers formed from 1 to 100 ethylene oxide units and from at least one fatty alcohol chain containing from 16 to 22 carbon atoms. The fatty chain of the ethers may be chosen especially from behenyl, arachidyl, stearyl, and cetyl units, and mixtures thereof, such as cetearyl. Examples of ethoxylated fatty ethers that may be mentioned include behenyl alcohol ethers comprising 5, 10, 20, and 30 ethylene oxide units (CTFA names: beheneth-5, beheneth-10, beheneth-20, beheneth-30), such as the products sold under the names Nikkol BB5, BB10, BB20, and BB30 by the company Nikko, and stearyl alcohol ether comprising 2 ethylene oxide units (CTFA name: steareth-2), such as the product sold under the name Brij 72 by the company ICI.

The ethoxylated fatty esters that are solid at a temperature equal to 45° C., which may be used as non-ionic amphiphilic lipids according to the invention are esters formed from 1 to 100 ethylene oxide units and from at least one fatty acid chain containing from 16 to 22 carbon atoms. The fatty chain of the esters may be chosen especially from stearate, behenate, arachidate, and palmitate units, and mixtures thereof. Examples of ethoxylated fatty esters that may be mentioned include stearic acid ester comprising 40 ethylene oxide units, such as the product sold under the name Myrj 52 (CTFA name: PEG-40 stearate) by the company ICI, and also the behenic acid ester comprising 8 ethylene oxide units (CTFA name: PEG-8 behenate), such as the product sold under the name Compritol HD5 ATO by the company Gattefosse.

5) The block copolymers of ethylene oxide and of propylene oxide that may be used as non-ionic amphiphilic lipids according to the invention may be chosen especially from the block copolymers of formula (V):
HO(C₂H₄O)ₓ (C₄H₆O)_{y}(C₂H₄O)_{z}H (V)
in which x, y, and z are integers such that x+z ranges from 2 to 100 and y ranges from 14 to 60, and mixtures thereof, and more particularly from the block copolymers of formula (V) with an HLB value ranging from 2 to 16.

These block copolymers may be chosen especially from poloxamers and especially from poloxamer 231, such as the product sold by the company ICI under the name Pluronic L81 of formula (V) with x=z=6, y=39 (HLB 2); poloxamer 282, such as the product sold by the company ICI under the name Pluronic L92 of formula (V) with x=z=10, y=47 (HLB 6); and poloxamer 124, such as the product sold by the company ICI under the name Pluronic L44 of formula (V) with x=z=11, y=21 (HLB 16).

Non-ionic amphiphilic lipids that may also be mentioned include the non-ionic surfactant mixtures described in document EP-A-705593, which is incorporated herein for reference.

Among the non-ionic amphiphilic lipids that may be used in particular are:
PEG-8 isostearate (comprising 8 mol of ethylene oxide),
triglyceryl hexadecyl ether,
sucrose distearate,
liquid diglyceryl isostearate,
polyglyceryl monolaurate comprising 2 glycerol units, and polyglyceryl stearates comprising from 2 to 10 glycerol units, and in particular those comprising 3 glycerol units,
liquid sorbitan oleate,
sorbitan palmitate,
sorbitan stearate comprising 4 mol of ethylene oxide, and mixtures thereof.

The non-ionic amphiphilic lipids may be present in the compositions according to the invention in a content ranging from 0.2% to 15% by weight, preferably ranging from 0.2% to 10% by weight and preferentially ranging from 0.2% to 8% by weight relative to the total weight of the composition.

The surfactants that are solid at a temperature of less than or equal to 45° C., chosen from fatty esters of glycerol, fatty esters of sorbitan and oxyethylenated fatty esters of sorbitan, ethoxylated fatty ethers, and ethoxylated fatty esters, are most particularly suitable as non-ionic amphiphilic lipids.

According to one particular embodiment of the invention, the compositions of the invention may also contain one or more ionic amphiphilic lipids, in particular one or more anionic or cationic lipids, other than the non-ionic amphiphilic lipids described previously.

Thus, the anionic amphiphilic lipids that may be used in the invention are preferably chosen from:
alkali metal salts of dicetyl and dimyristyl phosphate,
alkali metal salts of cholesteryl sulfate,
alkali metal salts of cholesteryl phosphate,
lipoamino acids and salts thereof, such as monosodium and disodium acylglutamates, for instance the disodium salt of N-stearoyl-L-glutamic acid sold under the name Acylglutamate HS21 by the company Ajinomoto,
the sodium salts of phosphatidic acid,
phospholipids,
alkylsulfonic derivatives especially of formula: 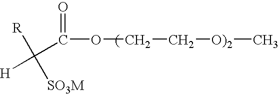
in which R represents C₁₆-C₂₂ alkyl radicals, in particular C₁₆H₃₃ and C₁₈H₃₇ radicals, taken as a mixture or separately, and M is an alkali metal or alkaline earth metal such as sodium; and mixtures thereof.

Lipoamino acids and salts thereof, especially those mentioned previously, are found to be particularly advantageous in the context of the invention.

The cationic amphiphilic lipids that may be used according to the invention are preferably chosen from the group formed by quaternary ammonium salts and fatty amines, and salts thereof.

As regards the quaternary ammonium salts, mention may be made more particularly of the salts having the general formula below: 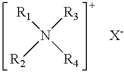
in which the radicals R₁ to R₄, which may be identical or different, represent a linear or branched aliphatic radical containing from 1 to 30 carbon atoms or an aromatic radical such as aryl or alkylaryl. The aliphatic radicals may comprise hetero atoms especially hetero atoms such as oxygen, nitrogen, sulfur, and halogens. The aliphatic radicals are chosen, for example, from alkyl, alkoxy, polyoxy(C₂-C₆)alkylene, alkylamide, (C₁₂-C₂₋₂)alkylamido(C₂-C₆)alkyl, (C₁₂-C₂₂)alkylacetate, and hydroxyalkyl, containing from about 1 to 30 carbon atoms; X is an anion chosen from the group of halides, phosphates, acetates, lactates, (C₂-C₆)alkyl sulfates, and alkyl- or alkylarylsulfonates.

Behenyltrimethylammonium chloride is most particularly suitable in the invention as quaternary ammonium salt.

A composition according to the invention may comprise from 0.01% to 6% by weight and preferably from 0.2% to 4% by weight of ionic amphiphilic lipid(s) relative to the total weight of the composition.

More generally, the total content of non-ionic and ionic amphiphilic lipids ranges from 0.25% to 18% by weight and preferably ranges from 1% to 10% by weight relative to the total weight of the composition.

Lamellar Liquid Crystal Phase

As mentioned previously, according to a first variant, a composition in accordance with the invention comprises a lipid phase organized as a lamellar liquid crystal phase.

In the context of the present invention, the term “lamellar liquid crystal phase” means a stack of swollen lipid bilayers.

A reference description is given in “The Colloidal Domain” second edition, by D. Fennell Evans and H. Wennerstrom, published by Wiley-VCH (1999), pages 295-296 and 306-307.

According to a first variant, this type of composition is an aqueous dispersion of lipid vesicles comprising a lamellar liquid crystal phase. The term “lipid vesicles (or spherules) comprising a lamellar liquid phase” means particles formed from a membrane consisting of one or more concentric leaflets, these leaflets comprising one or more bimolecular layers of amphiphilic lipids forming the lamellar liquid crystal phase encapsulating an aqueous phase. The aqueous phase may contain water-soluble active substances and the bimolecular layers of amphiphilic lipids may contain lipophilic active substances.

These vesicles generally have a mean diameter of between 10 and 5000 nanometres. Such aqueous dispersions of lipid vesicles, also known as liposomes or niosomes, are especially described in documents FR 2 532 191, FR 2 694 893, FR 2 714 596 and FR 2 730 931, the content of which is incorporated by reference.

The vesicles with an aqueous core according to the invention preferably comprise a lipid membrane formed from at least one non-ionic amphiphilic lipid and from at least one ionic amphiphilic lipid, which are swollen with a urea derivative of formula (I) as described previously.

The vesicle dispersions with an aqueous core may be prepared by any known process for manufacturing amphiphilic lipid vesicles and more particularly via the process known as “lipid cofusion”, which allows them to be prepared simply on an industrial scale.

According to a second embodiment of this variant, the compositions according to the invention comprise an oil-in-water emulsion formed from vesicles with an oily core having a lamellar liquid crystal phase coating, i.e. containing vesicles comprising a lipid membrane encapsulating an oily phase.

The vesicles with an oily core are preferably in the form of oily globules in dispersion individually coated with a monolamellar or oligolamellar layer (2 to 10 lipid leaflets), obtained from at least one non-ionic amphiphilic lipid with an HLB value of between 2 and 5 and from at least one non-ionic amphiphilic lipid with an HLB value of between 8 and 12.

Examples of surfactants with an HLB value ranging from 2 to 5 are sucrose distearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glyceryl ester of palmitic or stearic acid, polyoxyethylene 2 EO monosterate (comprising 2 oxyethylene units), glyceryl mono- and dibehenate, and pentaerythrityl tetrastearate.

Examples of surfactants with an HLB value ranging from 8 to 12 that may be mentioned include the following compounds: polyoxyethylenated sorbitan monostearate 4 EO, polyoxyethylenated sorbitan tristearate 20 EO, polyoxyethylenated monostearate 8 EO, hexaglyceryl monostearate, polyoxyethylenated monostearate 10 EO, polyoxyethylenated distearate 12 EO, and polyoxyethylenated methylglucose distearate 20 EO.

The lipid vesicles with an oily core in accordance with the invention may be prepared according to the manufacturing process described in patent applications FR 2709666 and FR 2725369. This process consists, in a first step, in mixing the fatty phase containing the non-ionic amphiphilic lipid with an HLB value of 2-5 and the amphiphilic lipid with an HLB value of 8-12, with stirring, and, in a second step, subjecting the mixture obtained to homogenization based on the cavitation principle. This homogenization is obtained either using high pressures of between 200 and 1500 bar, or using ultrasonication, or using homogenizers equipped with a rotor-stator head.

The mean size of the coated oily globules is less than 1000 nanometers and preferably less than or equal to 800 nanometers.

Such emulsions are especially described in documents FR 2 709 666, FR 2 725 369 and FR 2 767 691, the content of which is incorporated by reference.

According to a third embodiment, the compositions according to the invention are essentially anhydrous and comprise at least one fatty phase and at least one lamellar liquid crystal phase swollen with at least one urea derivative of formula (I) described previously.

Thus, it is known that certain mixtures of amphiphilic lipids known to form, by contact with an aqueous phase, vesicles consisting of more or less spherical leaflets of vesicular lipid phase encapsulating the aqueous phase, conserve this property of forming vesicles when they are in the form of a lamellar phase and are mixed with a fatty phase, generally used for the manufacture of essentially anhydrous cosmetic compositions, which contains organic and/or mineral oils, fatty substances, usually waxes, and surfactants.

Such compositions are especially described in document EP 534 823, the content of which is incorporated by reference.

The vesicles discussed previously are moreover particularly advantageous as vehicles.

Thus, the vesicles of the compositions according to the invention may contain, in a known manner, one or more active compounds with cosmetic and/or dermopharmaceutical activity, which, depending on their solubility characteristics, may have different localizations. If the active agents are water-soluble, they are preferably introduced into the encapsulated aqueous phase of the vesicles with an aqueous core. If the active agents are liposoluble, they are preferably introduced into the lipid phase constituting the membrane or into the encapsulated oily phase of the vesicles with an oily core, or else into the lamellar liquid crystal phase present in the essentially anhydrous composition.

If the active agents are amphiphilic, they are distributed between the lipid phase and the encapsulated aqueous phase of the vesicles with an aqueous core or between the lipid phase and the aqueous phase of the dispersion of vesicles with an oily core, with a partition coefficient that varies depending on the nature of the amphiphilic active agent and the respective compositions of the various phases in contact with the active agent.

The water-soluble active agents are, for example, glycerol, sorbitol, erythrulose, and antibiotics. The liposoluble or partially liposoluble (amphiphilic) active agents are chosen from those that do not significantly increase the permeability of the vesicles, do not cause them to flocculate and melt, and do not reduce their degree of encapsulation. Liposoluble active agents that also constitute additives are advantageously used.

The liposoluble active agents that are preferred according to the invention are chosen from the group formed by:

sphingomyelins,

glycoceramides, in particular those derived from wheatgerm, and

natural or synthetic ceramides, preferably those described in French patent application No. 2 673 179.

Moreover, it is possible, in a known manner, to incorporate into the lipid phase constituting the lipid membrane of the vesicles with an aqueous core at least one additive whose main function is to reduce the permeability of the vesicles, to prevent them from flocculating and melting, and to increase the degree of encapsulation.

At least one additive chosen especially from sterols and in particular phytosterols and cholesterol may thus be added to the lipid phase.

Cubic Liquid Crystal Phase

According to the second variant of the invention, the composition comprises an amphiphilic lipid phase organized in the form of a cubic liquid crystal phase. A cubic phase is organized in a bipolar manner into separate hydrophilic and lipophilic domains, which are in close contact and which form a thermodynamically stable three-dimensional network. Such an organization has been described especially in “La Recherche”, Vol. 23, pp. 306-315, March 1992 and in “Lipid Technology”, Vol. 2, No. 2, pp. 42-45, April 1990. Depending on the arrangement of the hydrophilic and lipophilic domains, the cubic phase is said to be of normal or inverse type. The term “cubic phase” used according to the present invention obviously includes the various types of cubic phases.

In general, the liquid crystal phases are described in “The aqueous phase behavior of surfactants” by R. G. Laughlin, published by Academic Press (1994), in chapter 8.4 pp. 200-237.

According to a first embodiment, this composition comprises a stable aqueous dispersion of cubic gel particles especially cubic gel particles simultaneously having hydrophilic and lipophilic domains.

Generally, the particles with a cubic liquid crystal phase are formed by fragmentation and dispersion of a gel with a cubic liquid crystal phase. These cubic liquid crystal phases may be obtained from amphiphilic lipids as defined previously, provided that they prove to be effective for leading to this type of architecture. Thus, certain amphiphilic lipids are capable of forming cubic phases when in the pure state, whereas others require the presence of a solvent (water or organic solvent) or an organic compound. Such gels may especially be prepared according to the protocol described in document WO 02/02716, the content of which is incorporated by reference.

Fragmentation of the gel is performed with a fragmenting agent. The fragmenting agent may be a hydrotrope, i.e. a compound capable of disrupting liquid crystal phases, as described in Pearson, J. T., Smith, J. M., “The effects of hydrotropic salts on the stability of liquid crystalline systems”, J. Pharm. Pharmac., 26, 123-124 (1974). The hydrotropes may be chosen from alcohols, polyols, ethoxylated alcohols, copolymers of ethylene oxide and of propylene oxide, and ethoxylated fatty acids. Hydrotropic compounds that may be mentioned in particular include ethanol, 1,4-butanediol, 1,2-hexanediol, ethylene glycol, propylene glycol, and glycerol. Such hydrotropic compounds are especially described in document WO-A-02/02716.

The fragmenting agent may also be chosen from amphiphilic polymers such as triblock block copolymers of polyethylene oxide/polypropylene oxide/polyethylene oxide sold under the name Pluronic by the company BASF, polyvinylpyrrolidone, amphiphilic proteins, for instance casein, and glycoproteins. The fragmenting agent may also be chosen from:
poloxyethylenated non-ionic surfactants, for instance oxyethylenated alkyl ethers, oxyethylenated alkyl esters, for instance alkyl esters of sorbitan polyoxyethylenated with at least 20 mol of ethylene oxide, such as sorbitan palmitate 20 EO sold under the name “Montanox 40 DF” by the company SEPPIC, sorbitan laurate 20 EO sold under the name “Tween 20” by the company SEPPIC, PEG-20 stearate, laureth-23 and oleth-20; oxyethylenated or non-oxyethylenated alkyl esters of polyglycerol, such as polyglyceryl-10 laurate sold under the name “Decaglyn 1-L” by the company Nikko Chemicals;
anionic surfactants, for instance alkyl ether sulfates such as sodium lauryl ether sulfate; alkyl ester sulfates such as isethionic acid esters and salts thereof and especially the sodium cocoyl isethionate sold under the name “Geropon AC 78” by the company Rhodia;
cationic surfactants, for instance alkyltrimethylammoniums, preferably those containing at least one C₁₂-C₂₂ alkyl group, for instance cocoyltrimethylammonium chloride and cetyltrimethylammonium bromide;
amphoteric surfactants, for instance alkyl betaines such as laurylamidopropyl betaine and oleylamidopropyl betaine.
   Such fragmenting agents are especially described in patent applications WO-A-93/06921 and FR-A-2720937.

Stable aqueous dispersions of cubic gel particles that may be mentioned more particularly include those based on 3,7,11,15-tetramethyl-1,2,3-hexadecanetriol or phytantriol. Phytantriol is a known compound sold especially under the name “Phytantriol-63926” by the company Roche. Such a dispersion is described especially in document FR 2 720 937, the content of which is incorporated by reference.

According to another embodiment, the composition according to the invention is in the form of a dispersion of an oily phase in an aqueous phase stabilized by such a cubic dispersion. Such a composition is described especially in document FR 2 726 762, the content of which is incorporated by reference.

Fatty Phase

In general, the compositions according to the invention also comprise a fatty phase and especially a liquid fatty phase.

Thus, the cosmetic compositions of the invention generally comprise at least one liquid fatty phase and especially at least one fatty substance that is liquid at room temperature (25° C.) and at atmospheric pressure and/or a fatty substance that is solid at room temperature and at atmospheric pressure. The fatty phase may also contain oil-gelling and oil-structuring agents of an organic nature and/or lipophilic organic solvents.

The fatty phase of the product according to the invention may especially comprise, as liquid fatty substance, at least one volatile or non-volatile oil, or a mixture thereof.

For the purposes of the invention, the term “volatile oil” means any oil capable of evaporating on contact with the skin or the lips in less than one hour, at room temperature and atmospheric pressure. The volatile oils of the invention are volatile cosmetic oils, which are liquid at room temperature, having a nonzero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” means an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, silicone oils, or mixtures thereof. The term “hydrocarbon-based oil” means an oil mainly containing hydrogen and carbon atoms and possibly oxygen, nitrogen, sulfur, and phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopar® and Permethyl®, branched C₈-C₁₆ esters such isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The non-volatile oils may be chosen especially from non-volatile hydrocarbon-based oils and, where appropriate, non-volatile fluoro and/or silicone oils. Non-volatile hydrocarbon-based oils that may especially be mentioned include:
hydrocarbon-based oils of animal origin
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810, 812 and 818′ by the company Dynamit Nobel,
synthetic ethers containing from 10 to 40 carbon atoms
linear or branched hydrocarbons of mineral or synthetic origin, such as petroleum jelly, polydecenes, hydrogenated polyisobutene such as parleam, and squalane, and mixtures thereof
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol
higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and mixtures thereof.

The liquid fatty phase and especially the oil or mixture of oils may represent from 0.01% to 99% of the total weight of the composition, in particular from 0.05% to 60% and more particularly from 2% to 40% by weight relative to the total weight of the composition.

The composition may comprise at least one solid fatty phase and in particular at least one wax. This solid fatty phase or the wax may represent from 0.01% to 70%, especially from 0.1% to 65% and in particular from 1% to 50% by weight relative to the total weight of the fatty phase.

The waxes that may be used in the invention are compounds that are solid at room temperature, intended to structure the composition, in particular in the form of a stick; they may be hydrocarbon-based waxes, fluoro waxes and/or silicone waxes and may be of plant, mineral, animal and/or synthetic origin. In particular, they have a melting point of greater than 40° C. and better still greater than 45° C.

With respect to waxes that may be used in the invention, mention may be made of those generally used in cosmetics: they are especially of natural origin, for instance beeswax, carnauba wax, candelilla wax, ouricury wax, Japan wax, cork fibre wax, sugarcane wax, rice wax, montan wax, paraffin, lignite wax or microcrystalline wax, ceresin, ozokerite and hydrogenated oils, for instance jojoba oil; synthetic waxes, for instance the polyethylene waxes derived from the polymerization or copolymerization of ethylene and Fischer-Tropsch waxes, or alternatively fatty acid esters, for instance octacosanyl stearate, glycerides that are solid at 40° C. and better still at 45° C., silicone waxes, for instance alkyl- or alkoxydimethicones containing an alkyl or alkoxy chain of 10 to 45 carbon atoms, poly(di)methylsiloxane esters that are solid at 40° C. and whose ester chain contains at least 10 carbon atoms; and mixtures thereof.

The compositions in accordance with the invention especially of aqueous dispersion type generally contain water and/or at least one water-soluble solvent. The term “water-soluble solvent” denotes a compound that is liquid at room temperature and miscible with water (water miscibility of greater than 50% by weight at 25° C. and atmospheric pressure).

The water-soluble solvents under consideration are also generally volatile. Among these water-soluble solvents, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones, and C₂ to C₄ aldehydes.

The compositions of the invention may also comprise at least one coloring agent that may be present especially in a proportion of from 0.01% to 40% by weight, especially from 0.01% to 30% by weight and in particular from 0.05% to 25% by weight relative to the total weight of the product.

This or these coloring agent(s) may be chosen from pigments, water-soluble or liposoluble dyes, nacres and flakes, and mixtures thereof.

The term “pigments” should be understood as meaning white or colored, mineral or organic particles, which are insoluble in the liquid hydrophilic phase and which are intended to color and/or opacify the composition. The term “nacres” should be understood as meaning iridescent particles, produced especially by certain molluscs in their shell, or alternatively synthesized.

Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate, and ferric blue, and metal powders, for instance aluminium powder or copper powder.

Among the organic pigments that may be mentioned are carbon black, pigments of D & C type, and lakes based on cochineal carmine or on barium, strontium, calcium, or aluminium.

Mention may also be made of pigments with an effect, such as particles comprising a natural or synthetic, organic or mineral substrate, for example glass, acrylic resins, polyester, polyurethane, polyethylene terephthalate, ceramics, or aluminas, the substrate being uncoated or coated with metal substances, for instance aluminium, gold, silver, platinum, copper, or bronze, or with metal oxides, for instance titanium dioxide, iron oxide, or chromium oxide, and mixtures thereof.

The nacreous pigments may be chosen from mica coated with titanium or with bismuth oxychloride, titanium mica coated with iron oxides, titanium mica coated especially with ferric blue or chromium oxide, titanium mica coated with an organic pigment of the abovementioned type, and also nacreous pigments based on bismuth oxychloride. Interference pigments, especially liquid-crystal pigments or multilayer pigments, may also be used.

Water-soluble dyes are, for example, beetroot juice or methylene blue.

The compositions according to the invention may furthermore comprise any ingredient conventionally used in the fields under consideration and more especially in cosmetics and dermatology. These ingredients are chosen in particular from vitamins, antioxidants, thickeners, trace elements, softeners, sequestering agents, fragrances, acidifying or basifying agents, preserving agents, UV-screening agents, and hydrophilic or lipophilic active agents, and mixtures thereof. The amounts of these various ingredients are those conventionally used in the fields under consideration, for example from 0.01% to 20% of the total weight of the composition.

In a known manner, the composition according to the invention may also contain active agents that are common in cosmetics or dermatology. Particular mention may be made of any active agent known for its activity on ageing of the skin, for instance keratolytic agents or pro-desquamating agents, for example α-hydroxy acids, β-hydroxy acids, α-keto acids, retinoids and esters thereof, retinol, retinoic acid and its derivatives. Mention may also be made of vitamins, for instance vitamin C, B3 or PP, B5, E, and derivatives of these vitamins and especially esters thereof; vitamin K and its derivatives (K1, K2, etc.); free-radical scavengers; sunscreens; moisturizers, for instance polyols; ceramides; DHEA and its derivatives; coenzyme Q10; bleaching and depigmenting agents, for instance kojic acid, paraaminophenol derivatives, and arbutin and derivatives thereof, and mixtures thereof.

The compositions according to the invention may be in the form of an emulsion or dispersion, especially oil-in-water (O/W) emulsions or dispersions. These compositions are prepared according to the usual methods. Preferably, the composition is an oil-in-water dispersion.

According to one variant of the invention, the composition is essentially anhydrous. It may then be in a form cast in stick form, for example in the case of a lipstick.

In addition, the compositions used according to the invention may be more or less fluid and may have the appearance of a white or colored cream, a pomade, a milk, a lotion, or a serum.

The composition of the invention may be used, for example, for caring for, treating, or making up keratin materials (especially human keratin materials), in particular the skin (especially of the face and/or the scalp), the lips, or the hair.

Thus, the composition according to the invention may be a skincare product, especially for the face, the neck, the contour of the eyes, or the body.

The makeup composition according to the invention may be a lip makeup product (lipstick), a foundation, a makeup rouge, an eyeshadow, an eyeliner, a concealer product, or a body makeup product.

The composition is advantageously a leave-on composition.

The composition of the invention may also be used for caring for and/or treating the hair.

A subject of the invention is thus also the cosmetic use of a composition as defined above for caring for and/or treating the hair.

The composition according to the invention especially allows good moisturization of the skin, mucous membranes, and/or the scalp, and is particularly suitable for treating dry skin.

Another subject of the invention is thus a cosmetic process for caring for, and/or moisturizing, and/or making up the skin or the lips, characterized in that a composition as defined above is applied to the skin or the lips.

The examples that follow are given as non-limiting illustrations of the present invention.

### EXAMPLE 1

### Mixture of Amphiphilic Lipids Swollen with N-(2-hydroxyethyl)urea to Form a Lamellar Liquid Crystal Phase

| | |
|---|---|
| Triglyceryl hexadecyl ether | 22.5% |
| Cholesterol | 22.5% |
| Dicetyl phosphate | 5.0% |
| N-(2-Hydroxyethyl)urea as an aqueous solution at | 50.0% |
| 50% by weight, sold by National Starch under the | |
| name Hydrovance ® | |

After melting the amphiphilic lipids at 110° C. and obtaining a homogeneous mixture, the temperature is reduced to 90° C. and the N-(2-hydroxyethyl)urea is added gradually, with stirring. After incorporation of all of the N-(2-hydroxyethyl)urea, a homogeneous white paste corresponding to a lamellar liquid crystal phase is obtained. When observed under an optical microscope, in polarization, an image in accordance with this type of structure is obtained (cf. “The aqueous phase behavior of surfactants” by R. G. Laughlin, published by Academic Press (1994), pages 539 and 560).

Thus, N-(2-hydroxyethyl)urea is at the polar heads of the amphiphilic lipids forming the lipid bilayers.

### EXAMPLE 2

### Care Product Based on Non-Ionic Liposomes Hydrated with N-(2-hydroxyethyl)urea

| Phase 1 | |
|---|---|
| Sorbitan palmitate (Span 40 ® - Uniqema) | 3.8% |
| Cholesterol | 3.8% |
| Monosodium salt of N-stearoyl-L-glutamic acid | 0.4% |
| (Acyglutamate HS 11 ® - Ajinomoto) | |
| Phase 2 | |
| N-(2-Hydroxyethyl)urea as an aqueous solution at | 10.0% |
| 50% by weight, sold by National Starch under the | |
| name Hydrovance ® | |
| Phase 3 | |
| Demineralized water | 45.0% |
| Preserving agents | 0.3% |
| Phase 4 | |
| Apricot kernel oil | 5.0% |
| Isocetyl stearate | 8.0% |
| Cyclomethicone (D5) | 10.0% |
| Phase 5 | |
| Hostacerin AMPS ® (Clariant) | 1.2% |
| Distilled water | 12.5% |

Phases 1 and 2 are mixed together as in Example 1. This mixture is then brought to 45° C. and then dispersed with very vigorous stirring (rotor-stator) in Phase 3. A suspension of non-ionic liposomes is thus obtained, which can then be homogenized using a high-pressure homogenizer (Niro-Soavi OBL20) at a pressure ranging from 20×10⁶ to 60×10⁶ pascals. Phase 4 is then dispersed with rotor-stator stirring in the dispersion 1+2+3 and, to optimize the dispersion, it is homogenized at between 20×10⁶ and 60×10⁶ pascals. A suspension of oil stabilized with non-ionic vesicles hydrated with hydroxyethylurea is thus obtained. After introduction of Phase 5, a moisturizing care emulsion is obtained, which is efficient and less tacky than the same composition containing glycerol instead of the hydroxyethylurea.

### EXAMPLE 3

### Lamellar Emulsion of Oleosome Type (Oily Globules Coated with a Lamellar Liquid Crystal Phase)

| Phase A1: | |
|---|---|
| Sucrose distearate (sold by the company | 2.0% |
| Stearinerie Dubois) | |
| Sorbitan stearate oxyethylenated with 4 mol of | 1.4% |
| ethylene oxide (sold under the name Tween 61 | |
| by the company ICI) | |
| Stearic acid | 0.75% |
| Stearyl heptanoate (sold by the company Dragoco | 5.50% |
| under the name PCL Solid ®) | |
| Petroleum jelly codex | 2.10% |
| Avocado oil | 4.50% |
| Jojoba oil | 4.10% |
| Volatile silicone oil | 3.70% |
| Vitamin E acetate | 0.50% |
| D-α-tocopherol (sold by the company Henkel | 0.30% |
| under the name Copherol 1300 ®) | |
| Phase A2: | |
| Silicone gum (sold by the company | 4.00% |
| Dow Corning under the name “Q2-1403 Fluid”) | |
| Fragrance | 0.3% |
| Propylparaben | 0.1% |
| Phase B: | |
| N-(2-Hydroxyethyl)urea as an aqueous solution at | 10.00% |
| 50% by weight (sold by National Starch under | |
| the name Hydrovance ®) | |
| Methylparaben | 0.30% |
| Triethanolamine | 0.40% |
| Demineralized water qs | 100.00% |
| Phase C: | |
| Mixture of carboxyvinyl polymers (sold under the | 0.30% |
| name Carbopol 980 by the company Goodrich) | |
| Demineralized water | 9.70% |

The two phases A1 and B are brought to 65° C. before being combined (B into A1) with very vigorous stirring (rotor-stator). After checking that the mixture is perfectly dispersed, it is optionally possible to homogenize the dispersion at between 20×10⁶ pascals and 60×10⁶ pascals, to obtain a dispersion whose oil globule size is less than 500 nm, the lamellar liquid crystal phase coating them having been hydrated with an aqueous hydroxyethylurea solution. Phase A2 is dispersed at room temperature into the first dispersion, with vigorous stirring. Phase C, prepared beforehand, is then dispersed in order to gel the suspension.

A moisturizing emulsion suitable for dry skin and less tacky than the same emulsion containing glycerol instead of Hydrovance® is obtained.

### EXAMPLE 4

### Stick of Lipstick Containing a Lamellar Liquid Crystal Phase Hydrated with N-(2-hydroxyethyl)urea

10% of the preparation given in Example 1 is introduced into a lipstick composition as described below.

Lipstick base:

| | |
|---|---|
| Polybutylene | 6.00% |
| Lanolin oil | 20.00% |
| Octoxyglyceryl behenate | 20.00% |
| Stearyl heptanoate | 9.80% |
| Castor oil | 17.00% |
| Jojoba oil | 9.80% |
| Antioxidant | 0.15% |
| Pigments | 7.25% |
| Lamellar liquid crystal phase of Example 1 | 10.00% |

Once the lipstick base has been prepared, at 100-110° C., the preparation from Example 1 is introduced at 80° C., with deflocculating stirring until a homogeneous colored base is obtained. The sticks are then cast. During application to the lips, non-ionic liposomes swollen with N-(2-hydroxyethyl)urea are formed, thus allowing the lips to be moisturized.

### EXAMPLE 5

### Care Cream Comprising a Dispersion of Cubic Gel Particles

An emulsion is prepared comprising a dispersion of cubic gel particles having the composition below:

| | |
|---|---|
| Phytantriol | 3 g |
| Hydroxyethylurea as an aqueous solution at 50% by weight | 1.2 g |
| (HYDROVANCE ® from the company National Starch) | |
| Preserving agent | 0.9 g |
| Distilled water qs | 100 g |
| Jojoba oil | 7.5 g |
| Petroleum jelly oil | 2.5 g |
| Cyclopentasiloxane | 10 g |

The aqueous hydroxyethylurea solution and the phytantriol are mixed together to obtain a compact cubic-phase transparent gel. This gel is then dispersed in the aqueous phase with vigorous stirring using a rotor-stator. High-pressure homogenization at 500 bar is performed to improve the homogeneity of the dispersion. The oily fraction is then added and dispersed using a rotor-stator. The mixture is homogenized again at a high pressure of 500 bar. A stable, sprayable, fluid suspension with an oil drop size of about 400 nm is obtained.

What is claimed is:

1. A cosmetic makeup and/or care composition, comprising:
at least one amphiphilic lipid phase organized as a lamellar or cubic liquid crystal phase, wherein the lipid phase comprises at least one urea-based compound of formula (I) 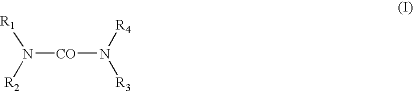
or a salt, solvate, or isomer thereof, wherein:
R₁, R₂, R₃, and R₄ each independently represent
a hydrogen atom,
a C₁-C₄ alkyl group, or
a C₂-C₆ hydroxyalkyl group that comprises from 1 to 5 hydroxyl groups;
wherein
at least one of the radicals R₁ to R₄ represents a hydroxyalkyl group,
said urea-based compound being present in the amphiphilic phase.

2. The composition according to claim 1, wherein R₁ denotes a C₂-C₆ hydroxyalkyl group and R₂, R₃, and R₄ each independently denotes a hydrogen atom or a C₁-C₄ alkyl group.

3. The composition according to claim 1, wherein R₁ denotes a C₂-C₆ hydroxyalkyl group comprising from 1 to 5 hydroxyl groups and R₂, R₃, and R₄ each denotes a hydrogen atom.

4. The composition according to claim 1, wherein R₁ denotes a C₂-C₆ hydroxyalkyl group comprising a hydroxyl group.

5. The composition according to claim 1, wherein R₁ denotes a C₂-C₄ hydroxyalkyl group comprising a hydroxyl group and R₂, R₃, and R₄ each denotes a hydrogen atom.

6. The composition according to claim 1, wherein the compound of formula (I) is selected from the group consisting of N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof.

7. The composition according to claim 1, wherein the compound of formula (I) is N-(2-hydroxyethyl)urea.

8. The composition according to claim 1, wherein the salt form is present and is a salt with an acid selected from the group consisting of: sulfuric acid, hydrochloric acid, hydrobromic acid, hydriodic acid, phosphoric acid, boric acid, propionic acid, acetic acid, terephthalic acid, citric acid, and tartaric acid.

9. The composition according to claim 1, wherein the compound of formula (I) is present in a content ranging from 1% to 50% by weight relative to the total weight of the composition.

10. The composition according to claim 1, wherein the amphiphilic lipid phase comprises at least one non-ionic amphiphilic lipid and an anionic amphiphilic lipid.

11. The composition according to claim 10, wherein the non-ionic amphiphilic lipid is selected from the group consisting of:
silicone surfactants;
esters of at least one polyol and of at least one fatty acid, the ester comprising at least one C₈-C₂₂ alkyl chain, wherein the polyol is:
a polyethylene glycol comprising from 1 to 60 ethylene oxide units,
sorbitan,
a glycerol comprising from 2 to 30 ethylene oxide units, or
a polyglycerol comprising from 2 to 15 glycerol units,
wherein the ester is a liquid at a temperature equal to 45° C.;
fatty acid esters of sugars;
fatty alkyl ethers of sugars;
surfactants that are solid at a temperature equal to 45° C., chosen from fatty esters of glycerol, fatty esters of sorbitan, oxyethylenated fatty esters of sorbitan, ethoxylated fatty ethers, or ethoxylated fatty esters;
block copolymer of ethylene oxide (A) and of propylene oxide (B);
phospholipids; and
mixtures thereof.

12. The composition according to claim 1, wherein the composition comprises from at least 0.2% to 15% by weight of non-ionic amphiphilic lipid.

13. The composition according to claim 1, further comprising at least one ionic amphiphilic lipid.

14. The composition according claim 13, wherein the ionic amphiphilic lipid is selected from the group consisting of:
alkali metal salts of dicetyl and dimyristyl phosphate;
alkali metal salts of cholesteryl sulfate;
alkali metal salts of cholesteryl phosphate;
lipoamino acids and salts thereof;
the sodium salts of phosphatidic acid;
phospholipids;
alkylsulfonic derivatives;
quaternary ammonium salts;
fatty amines and salts thereof; and
mixtures thereof.

15. The composition according to claim 1, wherein the composition comprises from 0.01% to 6% by weight of ionic amphiphilic lipid.

16. The composition according to claim 1, wherein the total content of non-ionic and ionic amphiphilic lipids ranges from 0.25% to 18% by weight.

17. The composition according to claim 1, further comprising a lipid phase organized as a lamellar liquid crystal phase.

18. The composition according to claim 17, wherein the composition comprises an aqueous dispersion of lipid vesicles comprising said lamellar lipid crystal phase.

19. The composition according to claim 17, wherein the composition is anhydrous and further comprises a fatty phase separate from the lamellar liquid crystal phase.

20. The composition according to claim 18, wherein the vesicles are liposomes or niosomes.

21. The composition according to claim 17, wherein the composition is in the form of an oil-in-water emulsion comprising vesicles with an oily core having a lamellar liquid crystal phase coating.

22. The composition according to claim 1, further comprising a stable aqueous dispersion of cubic gel particles.

23. The composition according to claim 22, wherein the composition is in the form of an oil-in-water dispersion stabilized by the dispersion of cubic gel particles.

24. The composition of claim 1, further comprising a liquid fatty phase.

25. The composition of claim 1, further comprising at least one coloring agent.

26. A cosmetic process for treating keratin material, wherein the composition according claim 1 is applied to the keratin material.

27. A cosmetic skin makeup process comprising applying to the skin a composition according to claim 1.
